(19) United States
(12) Patent Application Publication
(10) Pub. No.: US 2021/0094920 A1
(43) Pub. Date: Apr. 1, 2021

(54) NR2B LIGANDS; METHOD OF MAKING; AND USE THEREOF

(71) Applicant: THE UNITED STATES OF AMERICA, AS REPRESENTED BY THE SECRETARY, DEPARTMENT OF HEALTH AND HUMAN SERVIC, Bethesda, MD (US)

(72) Inventors: Lisheng Cai, Derwood, MD (US); Victor W. Pike, Bethesda, MD (US); Robert B. INNIS, Bethesda, MD (US)

(21) Appl. No.: 17/050,209

(22) PCT Filed: Apr. 26, 2019
(86) PCT No.: PCT/US19/29285

§ 371 Date: Oct. 23, 2020

Related U.S. Application Data
(60) Provisional application No. 62/663,085, filed on Apr. 26, 2018.

(51) Int. Cl. C07D 223/16; A61K 51/04; C07D 401/06; C07D 405/06

(52) U.S. Cl. CPC C07D 223/16; C07D 405/06; C07D 401/06; A61K 51/0468

(57) ABSTRACT

Disclosed herein are derivatives of 7-methoxy-3-(4-phenylbutyl)-2,3,4,5-tetrahydro-1H-benzo[d]azepin-1-ol, including radiolabeled derivatives, which are found to be NR2B receptor subunit ligands. Pharmaceutical compositions comprising the derivative compounds and methods of treating schizophrenia, depression, stroke, or a neurodegenerative disease in a subject are also disclosed.

### FIELD OF THE DISCLOSURE

The present disclosure is directed to derivatives of 7-methoxy-3-(4-phenylbutyl)-2,3,4,5-tetrahydro-1H-benzo[d]azepin-1-ol, including radiolabeled derivatives, which are found to be NR2B ligands.

### BACKGROUND

Noninvasive, nuclear imaging techniques can be used to obtain basic and diagnostic information about the physiology and biochemistry of living subjects in general, including experimental animals, normal humans and patients. Positron emission tomography (PET) relies on the use of imaging instruments that can detect radiation emitted from radiotracers administered to living subjects. The information obtained can be reconstructed to provide planar and tomographic images that reveal the distribution and/or concentration of the radiotracer as a function of time.

PET is a noninvasive imaging technique that offers the highest spatial and temporal resolution of all nuclear medicine imaging modalities and has the added advantage that it can allow for true quantitation of tracer concentrations in tissues. The technique involves the use of radiotracers, labeled with positron-emitting radionuclides, that are designed to have in vivo properties that permit measurement of parameters regarding the physiology or biochemistry of a variety of processes in living tissue.

Radiotracers can be labeled with positron-emitting radionuclides. The most commonly used positron-emitting radionuclides are ¹⁵O, ¹³N, ¹¹C and ¹⁸F, which are usually accelerator-produced and have a half-life of 2, 10, 20 and 110 minutes, respectively.

The NR2B is the most studied N-methyl-D-aspartate (NMDA) receptor subunit within the NMDA complex, and its expression is largely limited to forebrain regions and dorsal horn of the spinal cord. NR2B is considered to be a therapeutic target for schizophrenia, stroke, and neurodegenerative diseases, especially neuropain. Therapeutics targeting NR2B rather than the NMDA channel have fewer side-effects. The quantification of NR2B subunits within NMDA receptors could help to elucidate the contribution of this receptor to neuropsychiatric disorders and also assist in drug development. Currently, no PET radioligand is available for such quantification. Thus, there remains a need in the art for novel, selective therapeutic compounds targeting NR2B and novel radiolabeled compounds selective for NR2B.

### SUMMARY

In an embodiment, is a compound of Formula I or a radioligand thereof, and/or a pharmaceutically acceptable salt thereof:

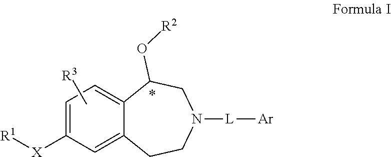

wherein

X is O or S, specifically O;

R¹ is H, C₁-C₆ alkyl, C₁-C₆ haloalkyl, specifically C₁-C₃ alkyl, and more specifically methyl;

R² is H, C₁-C₆ alkyl, Q-C₆ haloalkyl, specifically H or C₁-C₃ alkyl, and more specifically H;

R³ is H, C₁-C₆ alkyl, C₁-C₆ alkoxy, C₁-C₆ haloalkyl, C₁-C₆ haloalkoxy, —OH, halo, —COOH, —CN, —NO₂, amino, mono- or di-alkylamino, C₂-C₆ alkanoyl, C₂-C₈ cycloalkyl, or heterocycloalkyl, specifically H, C₁-C₃ alkyl, or C₁-C₃ haloalkyl, and more specifically H;

L is a linking group; and

Ar is an aryl or heteroaryl group, each of which is optionally substituted with one, two, or three substituents;

with the provision that the compound is not 3-(4-phenylbutyl)-2,3,4-tetrahydro-H-benzo[d]azepine-1,7-diol; 7-methoxy-3-(4-phenylbutyl)-2,3,4,5-tetrahydro-H-benzo[d]azepin-1-ol; or 7-[¹¹C]methoxy-3-(4-phenylbutyl)-2,3,4,5-tetrahydro-1H-benzo[d]azepin-1-ol. The “*” indicates a stereogenic center. In an embodiment the stereogenic center * is racemic. In an embodiment the stereogenic center * is in the R configuration. In an embodiment the stereogenic center * is in the S configuration.

In an embodiment, a pharmaceutical composition comprises a compound of Formula I or a salt thereof and at least one pharmaceutically acceptable carrier.

In an embodiment, a method for the treatment of schizophrenia, depression, stroke, or a neurodegenerative disease, especially neuropain, comprises providing a therapeutically effective amount of a compound of Formula I or salt thereof to a patient in need of such treatment.

In another embodiment, a method for quantifying NR2B receptor subunits within NMDA receptors in a subject comprises, administering a radiolabeled compound of Formula I to a subject, and quantifying the concentration of the radiolabeled compound using positron emission tomography.

### BRIEF DESCRIPTION OF DRAWINGS

FIG. 1: illustrates the measured Ki vs. clogD follows an exponential curve for the synthesized MTB derivatives.

FIG. 2: illustrates displacement of [¹¹C]NR2B-Me in rat by Ro 25 6981 at different doses.

FIG. 3: whole brain PET time activity curves in rats at baseline, in rats pretreated with various agents, and in rats given NR2B-SMe or eliprodil after radioligand.

FIG. 4: areas under the brain time-activity curve (AUC) between 50 and 100 min for the PET experiments in FIG. 3.

FIG. 5: dependence of AUC on dose of NR2B-SMe1 and SA4503.

FIG. 6: illustrates displacement of [¹¹C]NR2B-MeI in rat by Ro 25 6981 at different doses.

FIG. 7: illustrates the dependence of AUC for [¹¹C]NR2B-MeI on the dose of Ro 25 6981.

### DETAILED DESCRIPTION

Disclosed are derivatives of Formula I based on 7-methoxy-2,3,4,5-tetrahydro-1H-benzo[d]azepin-1-ol (MTB), including radiolabeled derivatives, which show specific binding with the NR2B receptor within the NMDA complex in the neuro-system.

A compound of Formula I or a radioligand thereof, and/or a pharmaceutically acceptable salt thereof:

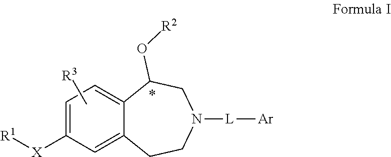

wherein X is O or S, specifically O;
R¹ is H, C₁-C₆ alkyl, C₁-C₆ haloalkyl, specifically C₁-C₃ alkyl, and more specifically methyl;
R² is H, C₁-C₆ alkyl, C₁-C₆ haloalkyl, specifically H or C₁-C₃ alkyl, and more specifically H;
R³ is H, C₁-C₆ alkyl, C₁-C₆ alkoxy, C₁-C₆ haloalkyl, C₁-C₆ haloalkoxy, —OH, halo, —COOH, —CN, —NO₂, amino, mono- or di-alkylamino, C₂-C₆ alkanoyl, C₂-C₈ cycloalkyl, or heterocycloalkyl, specifically H, C₁-C₃ alkyl, or C₁-C₃ haloalkyl, and more specifically H;
L is a linking group; and
Ar is an aryl or heteroaryl group, each of which is optionally substituted with one, two, or three substituents as defined herein.

When the compound of Formula I is a radioligand, an atom selected from carbon, hydrogen, nitrogen, oxygen and halogen atom comprises, or is replaced by, a detectable amount of a radioisotope. The radioisotope can be ¹¹C, ¹³N, ¹⁵O, ¹⁸F, ⁷⁵Br, or ⁷⁶Br.

In an embodiment, the linking group L of Formula I is an optionally substituted divalent C₂-C₈ alkyl, C₂-C₈ alkenyl, or C₂-C₈ alkynyl, specifically a divalent C₂-C₆ alkyl, C₂—C; alkenyl, or C₂—C alkynyl, more specifically a divalent C₂-C₄ alkyl, C₂-C₄ alkenyl, or C₂-C₄ alkynyl, and yet more specifically a divalent C₃-C₄ alkyl. When optionally substituted, the substituents can be pendent from the linking group chain or substituted with one, two, or three heteroatoms between carbon atoms within linking group chain (e.g. with O to form an ether), the heteroatoms selected from O, S, S═O, S(═O)₂, or NR wherein R is —I, C₁-C₆ alkyl, C₁-C₆ haloalkyl, or C₂-C₆ alkanoyl. Suitable pendent substituents include oxo C₁-C₆ alkyl, C₁-C₆alkoxy, C₁-C₆ haloalkyl, C₁-C₆ haloalkoxy, —OH, halo, —COOH, —CN, —NO₂, amino, mono- or di-alkylamino C₁-C₆ alkanoyl, C₂-C₈ cycloalkyl, or heterocycloalkyl.

In an embodiment, the Ar group of Formula I is phenyl, naphthyl, bi-phenyl, pyridyl, benzofuranyl, coumarinyl, quinolinyl, isoquinolinyl, quinazolinyl, pyridizinyl, pyrazinyl, pyrimidinyl, furanyl, oxazolyl, pyrrolyl, thienyl, thiazolyl, triazinyl, triazolyl, tetrazolyl, isoxazolyl, imidazolyl, indolyl, benz[b]thiophenyl benzothiazolyl, pyrazolyl, isoquinolinyl, quinazolinyl, quinoxalinyl, or isoindolyl, each of which is optionally substituted with one, two, or three substituents individually selected from C₁-C₆ alkyl, C₁-C₆ alkoxy, C₁-C₆ thioalkyl, C₁-C₆ haloalkyl, C₁-C₆ haloalkoxy, —OH, halo, —COOH, —CN, —NO₂, amino, mono- or di-alkylamino, C₂-C₆ alkanoyl, C₂-C₈ cycloalkyl, heterocycloalkyl, (C₁-C₆ alkoxycarbonyi)C₀-C₆ alkyl, (C₁-C₆ alkoxycarbonyl)C₀-C₆ alkoxy, or (C₁-C₆ alkoxycarbonyl)C₀-C₆ thioalkyl groups, specifically C₁-C₆ alkyl, C₁-C₆ alkoxy, C₁-C₆ thioalkyl, C₁-C₆ haloalkyl, halo, or (C₁-C₆alkoxycarbonyl)C₀-C₆ thioalkyl. For example, (C₁-C₆ alkoxycarbonyO)C₂ thioalkyl group is —S—CH₂CH₂(C═O)OCH₃ which is linked to the Ar group through the S atom.

The binding affinities of the compounds of Formula I can be adjusted by modifying the lipophilicity of the compound, modifying the linking group (L) separating the benzo[d]azepine moiety and the Ar group, and by modifying the substituents on the Ar group. Compounds have been synthesized exhibiting lower nanomolar binding affinity to the NR2B receptor.

Also included in this disclosure are compounds of Formula I, as set out in Table 1 of the Examples (excluding compound 1), specifically compounds 2-15 and their radiolabeled derivatives thereof. Within this embodiment, the radiolabeled derivatives of compounds 2-15 have a radioisotope, specifically [¹¹C] atom, located at the Ar group. In another embodiment, the radiolabeled derivatives of compounds 2-15 have a [¹¹C]O— group at the 7-position of the 2,3,4,5-tetrahydro-1H-benzo[d]azepin-1-ol core.

In an embodiment, the derivative of Formula I is 7-methoxy-3-(4-(4-methylphenyl)butyl)-2,3,4,5-tetrahydro-1H-benzo[d]azepin-1-ol (NR2B-Me), 7-methoxy-3-(4-(4-(methylthio)phenyl)butyl)-2,3,4,5-tetrahydro-1H-benzo[d]azepin-1-ol (NR2B-SMe), or a radiolabeled derivatives thereof. Specifically, NR2B-Me shows affinity for NR2B in the nM range, moderate computed lipophilicity (clogD=3.4), and amenability to labeling with carbon-1 to [¹¹C]NR2B-Me. Similarly, NR2B-SMe shows affinity for NR2B in the nM range, moderate computed lipophilicity (clogD=3.7), and amenability to labelling with carbon-11.

[¹¹C]NR2B-Me can be prepared from a boronic ester precursor and NR2B-SMe can be prepared from an S-methyl propionate precursor as described in the Examples.

The compounds are described using standard nomenclature. Unless defined otherwise, all technical and scientific terms used herein have the same meaning as is commonly understood by one of skill in the art to which this disclosure belongs. Unless clearly contraindicated by the context each compound name includes the free acid or free base form of the compound as well hydrates of the compound and all pharmaceutically acceptable salts of the compound.

The term “Formula I”, as used herein, encompasses all compounds that satisfy Formula I, including any enantiomers, racemates and stereoisomers, as well as all pharmaceutically acceptable salts of such compounds. The phrase “a compound of Formula I” includes all subgeneric groups of Formula I, as well as all forms of such compounds, including salts, hydrates, and radiolabeled forms unless clearly contraindicated by the context in which this phrase is used.

In certain situations, the compounds of Formula I may contain one or more asymmetric elements such as stereogenic centers, stereogenic axes and the like, e.g. asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In these situations, single enantiomers, i.e., optically active forms, can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example, a chiral HPLC column.

Where a compound exists in various tautomeric forms, the compound is not limited to any one of the specific tautomers, but rather includes all tautomeric forms.

Certain compounds are described herein using a general formula that includes variables, e.g. R¹, R², etc. Unless otherwise specified, each variable within such a formula is defined independently of other variables. Thus, if a group is said to be substituted, e.g., with 0-2 R^{x}, then the group may be substituted with up to two R groups and R at each occurrence is selected independently from the definition of R^{x}. Also, combinations of substituents and/or variables are permissible only if such combinations result in stable compounds.

A dash (“-”) that is not between two letters or symbols is used to indicate a point of attachment for a substituent. For example, —(CH₂)C₃-C₈ cycloalkyl is attached through carbon of the methylene (CH₂) group.

“Alkanoyl” is an alkyl group as defined herein, covalently bound to the group it substitutes by a keto (—(C═O)—) bridge. Alkanoyl groups have the indicated number of carbon atoms, with the carbon of the keto group being included in the numbered carbon atoms. For example a C alkanoyl group is an acetyl group having the formula CH₃(C═O)—.

The term “alkyl”, as used herein, means a branched or straight chain saturated aliphatic hydrocarbon group having the specified number of carbon atoms, generally from 1 to about 12 carbon atoms. The term C₁-C₆ alkyl as used herein indicates an alkyl group having from 1, 2, 3, 4, 5, or 6 carbon atoms. Other embodiments include alkyl groups having from 1 to 8 carbon atoms, 1 to 4 carbon atoms 1 or 2 carbon atoms, e.g. C₁-C₆ alkyl, C₁-C₄ alkyl, and C₁-C₂ alkyl. When C₀-Cₙ alkyl is used herein in conjunction with another group, for example, (cycloalkyl)C₀-C₄ alkyl, the indicated group, in this case cycloalkyl, is either directly bound by a single covalent bond (Co), or attached by an alkyl chain having the specified number of carbon atoms, in this case 1, 2, 3, or 4 carbon atoms. Examples of alkyl include, but are not limited to, methyl, ethyl, n-propyl, isopropyl, n-butyl, 3-methylbutyl, t-butyl, n-pentyl, and sec-pentyl.

The term “cycloalkyl”, as used herein, indicates a saturated hydrocarbon ring group, having only carbon ring atoms and having the specified number of carbon atoms, usually from 3 to about 8 ring carbon atoms, or from 3 to about 7 carbon atoms. Examples of cycloalkyl groups include cyclopropyl, cyclobutyl, cyclopentyl, or cyclohexyl as well as bridged or caged saturated ring groups such as norborane or adamantane.

The term “heterocycloalkyl”, as used herein, indicates a saturated cyclic group containing from 1 to about 3 heteroatoms chosen from N, O, and S, with remaining ring atoms being carbon. Heterocycloalkyl groups have from 3 to about 8 ring atoms, and more typically have from 5 to 7 ring atoms. Examples of heterocycloalkyl groups include morpholinyl, piperazinyl, piperidinyl, and pyrrolidinyl groups. A nitrogen in a heterocycloalkyl group may optionally be quaternized.

The term “alkenyl” as used herein, means straight and branched hydrocarbon chains comprising one or more unsaturated carbon-carbon bonds, which may occur in any stable point along the chain. Alkenyl groups described herein typically have from 2 to about 12 carbon atoms. Exemplary alkenyl groups are lower alkenyl groups, those alkenyl groups having from 2 to about 8 carbon atoms, e.g. C₂-C₈, C₂-C₆, and C₂-C₄ alkenyl groups. Examples of alkenyl groups include ethenyl, propenyl, and butenyl groups.

The term “alkynyl”, means straight and branched hydrocarbon chains comprising one or more C≡C carbon-carbon triple bonds, which may occur in any stable point along the chain. Alkynyl groups described herein typically have from 2 to about 12 carbon atoms. Exemplary alkynyl groups are lower alkynyl groups, those alkenyl groups having from 2 to about 8 carbon atoms, e.g. C₂-C₈, C₂-C₆, and C₂-C₄ alkynyl groups. Examples of alkynyl groups include ethynyl, propynyl, and butynyl groups.

The term “cycloalkenyl”, as used herein, means a saturated hydrocarbon ring group, comprising one or more unsaturated carbon-carbon bonds, which may occur in any stable point of the ring, and having the specified number of carbon atoms. Monocyclic cycloalkenyl groups typically have from 3 to about 8 carbon ring atoms or from 3 to 7 (3, 4, 5, 6, or 7) carbon ring atoms. Cycloalkenyl substituents may be pendant from a substituted nitrogen or carbon atom, or a substituted carbon atom that may have two substituents may have a cycloalkenyl group, which is attached as a spiro group. Examples of cycloalkenyl groups include cyclopropenyl, cyclobutenyl, cyclopentenyl, or cyclohexenyl as well as bridged or caged saturated ring groups such as norbornene.

The terms “(cycloalkyl)Co-C alkyl”, as used herein, means a substituent in which the cycloalkyl and alkyl are as defined herein, and the point of attachment of the (cycloalkyl)alkyl group to the molecule it substitutes is either a single covalent bond, (C₀alkyl) or on the alkyl group. (Cycloalkyl)alkyl encompasses, but is not limited to, cyclopropylmethyl, cyclobutylmethyl, and cyclohexylmethyl.

The terms “(heterocycloalkyl)C₀-Cₙ alkyl”, as used herein, means a substituent in which the heterocycloalkyl and alkyl are as defined herein, and the point of attachment of the (heterocycloalkyl)alkyl group to the molecule it substitutes is either a single covalent bond, (C alkyl) or on the alkyl group. (Heterocycloalkyl)alkyl encompasses, but is not limited to, morpholinylmethyl, piperazinylmethyl, piperidinylmethyl, and pyrrolidinylmethyl groups.

The term “aryl”, as used herein, means aromatic groups containing only carbon in the aromatic ring or rings. Typical aryl groups contain 1 to 3 separate, fused, or pendant rings and from 6 to about 18 ring atoms, without heteroatoms as ring members. When indicated, such aryl groups may be further substituted with carbon or non-carbon atoms or groups. Bicyclic aryl groups may be further substituted with carbon or non-carbon atoms or groups. Bicyclic aryl groups may contain two fused aromatic rings (naphthyl) or an aromatic ring fused to a 5- to 7-membered non-aromatic cyclic group that optionally contains 1 or 2 heteroatoms independently chosen from N, O, and S, for example, a 3,4-methylenedioxy-phenyl group. Aryl groups include, for example, phenyl, naphthyl, including 1-naphthyl and 2-naphthyl, and bi-phenyl.

The term “mono- or bicyclic heteroaryl”, as used herein, indicates a stable 5- to 7-membered monocyclic or 7- to 10-membered bicyclic heterocyclic ring which contains at least 1 aromatic ring that contains from 1 to 4, or specifically from 1 to 3, heteroatoms chosen from N, O, and S, with remaining ring atoms being carbon. When the total number of S and O atoms in the heteroaryl group exceeds 1, theses heteroatoms are not adjacent to one another. Specifically, the total number of S and O atoms in the heteroaryl group is not more than 2, more specifically the total number of S and O atoms in the heteroaryl group is not more than 1. A nitrogen atom in a heteroaryl group may optionally be quaternized. When indicated, such heteroaryl groups may be further substituted with carbon or non-carbon atoms or groups. Such substitution may include fusion to a 5 to 7-membered saturated cyclic group that optionally contains 1 or 2 heteroatoms independently chosen from N, O, and S, to form, for example, a [1,3]dioxolo[4,5-c]pyridyl group. In certain embodiments 5- to 6-membered heteroaryl groups are used. Examples of heteroaryl groups include, but are not limited to, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, benz[b]thiophenyl, isoquinolinyl, quinazolinyl, quinoxalinyl, thienyl, isoindolyl, and 5,6,7,8-tetrahydroisoquinoline.

“Haloalkyl” includes both branched and straight-chain alkyl groups having the specified number of carbon atoms, substituted with 1 or more halogen atoms, up to the maximum allowable number of halogen atoms. Examples of haloalkyl include, but are not limited to, trifluoromethyl, difluoromethyl, 2-fluoroethyl, and penta-fluoroethyl.

“Haloalkoxy” is a haloalkyl group as defined herein attached through an oxygen bridge (oxygen of an alcohol radical).

“Halo” or “halogen” is any of fluoro, chloro, bromo, and iodo.

“Mono- and/or di-alkylamino” is a secondary or tertiary alkyl amino group, wherein the alkyl groups are independently chosen alkyl groups, as defined herein, having the indicated number of carbon atoms. The point of attachment of the alkylamino group is on the nitrogen. Examples of mono- and di-alkylamino groups include ethylamino, dimethylamino, and methyl-propyl-amino. Amino means —NH₂.

The term “substituted”, as used herein, means that any one or more hydrogens on the designated atom or group is replaced with a selection from the indicated group, provided that the designated atom's normal valence is not exceeded. When the substituent is oxo (i.e., ═O) then 2 hydrogens on the atom are replaced. When an oxo group substitutes aromatic moieties, the corresponding partially unsaturated ring replaces the aromatic ring. For example, a pyridyl group substituted by oxo is a pyridone. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

Unless otherwise specified substituents are named into the core structure. For example, it is to be understood that when (cycloalkyl)alkyl is listed as a possible substituent the point of attachment of this substituent to the core structure is in the alkyl portion, or when arylalkyl is listed as a possible substituent the point attachment to the core structure is the alkyl portion.

Suitable groups that may be present on a “substituted” or “optionally substituted” position include, but are not limited to, halogen; cyano; hydroxyl; nitro; azido; alkanoyl (such as a C₂-C₆ alkanoyl group such as acyl or the like); carboxamido; alkyl groups (including cycloalkyl groups) having 1 to about 8 carbon atoms, or 1 to about 6 carbon atoms; alkenyl and alkynyl groups including groups having one or more unsaturated linkages and from 2 to about 8, or 2 to about 6 carbon atoms; alkoxy groups having one or more oxygen linkages and from 1 to about 8, or from 1 to about 6 carbon atoms; aryloxy such as phenoxy; alkylthio groups including those having one or more thioether linkages and from 1 to about 8 carbon atoms, or from 1 to about 6 carbon atoms; alkylsulfinyl groups including those having one or more sulfinyl linkages and from 1 to about 8 carbon atoms, or from 1 to about 6 carbon atoms; alkylsulfonyl groups including those having one or more sulfonyl linkages and from 1 to about 8 carbon atoms, or from 1 to about 6 carbon atoms; aminoalkyl groups including groups having one or more N atoms and from 1 to about 8, or from 1 to about 6 carbon atoms; aryl having 6 or more carbons and one or more rings, (e.g., phenyl, biphenyl, naphthyl, or the like, each ring either substituted or unsubstituted aromatic); arylalkyl having 1 to separate or fused rings and from 6 to about 18 ring carbon atoms, with benzyl being an exemplary arylalkyl group; arylalkoxy having 1 to 3 separate or fused rings and from 6 to about 18 ring carbon atoms, with benzyloxy being an exemplary arylalkoxy group; or a saturated, unsaturated, or aromatic heterocyclic group having 1 to 3 separate or fused rings with 3 to about 8 members per ring and one or more N, O or S atoms, e.g. coumarinyl, quinolinyl, isoquinolinyl, quinazolinyl, pyridyl, pyrazinyl, pyrimidinyl, furanyl, pyrrolyl, thienyl, thiazolyl, triazinyl, oxazolyl, isoxazolyl, imidazolyl, indolyl, benzofuranyl, benzothiazolyl, tetrahydrofuranyl, tetrahydropyranyl, piperidinyl, morpholinyl, piperazinyl, and pyrrolidinyl. Such heterocyclic groups may be further substituted, e.g. with hydroxy, alkyl, alkoxy, halogen and amino.

Compounds of Formula I having a carbon-11 may be labeled by the use of [¹¹C]carbon monoxide insertion reactions.

Compounds of Formula I having ¹⁸F in their structures may be labeled, for example, by already known general methods for introducing [¹⁸F]fluoride at aryl rings, including aromatic nucleophilic substitution of leaving groups (e.g. NO), halo, R₃N⁺) and reactions of [¹⁸F]fluoride ion with diaryl iodonium salts, iodonium ylides, sulfoxides, selenoxides, or boronic acid esters.

The term “pharmaceutically acceptable salt”, as used herein, includes derivatives of the disclosed compounds in which the parent compound is modified by making inorganic and organic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are used, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in Remington's Pharmaceutical Sciences, 17th ed., Mack Publishing Company, Easton, Pa., p. 1418 (1985).

The term “active agent”, as used herein, means a compound (including a compound of Formula I, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound, crystalline forms, non-crystalline forms, and any polymorphs of the compound are included. All forms are contemplated herein regardless of the methods used to obtain them.

Also provided are pharmaceutical compositions comprising a compound of Formula I and a pharmaceutically acceptable carrier. Such pharmaceutical compositions may contain a compound of Formula I as the only active agent or may contain a combination of a compound of Formula I and another pharmaceutically active agent. Also provided is a method for the treatment of schizophrenia, depression, stroke, or a neurodegenerative disease, especially neuropain, comprising providing a therapeutically effective amount of a compound of Formula I or salt thereof to a patient in need of such treatment.

The term “dosage form”, as used herein, means a unit of administration of an active agent. Examples of dosage forms include tablets, capsules, injections, suspensions, liquids, emulsions, creams, ointments, suppositories, inhalable forms, transdermal forms, and the like. Exemplary dosage form is a solid oral dosage form.

The term “pharmaceutical compositions”, as used herein, are compositions comprising at least one active agent, such as a compound or salt of Formula I, and at least one other substance, such as a carrier. Pharmaceutical compositions meet the U.S. FDA's GMP (good manufacturing practice) standards for human or non-human drugs. The pharmaceutical compositions can be formulated into a dosage form.

The term “carrier”, as used herein, applied to pharmaceutical compositions refers to a diluent, excipient, or vehicle with which an active compound is provided.

The term “patient”, as used herein, is a human or non-human animal in need of medical treatment. Medical treatment can include treatment of an existing condition, such as a disease or disorder, prophylactic or preventative treatment, or diagnostic treatment. In some embodiments the patient is a human patient.

The term “providing”, as used herein, means giving, administering, selling, distributing, transferring (for profit or not), manufacturing, compounding, or dispensing.

The term “providing a compound of Formula I with at least one additional therapeutic agent”, as used herein, means the compound of Formula I and the additional active agent(s) are provided simultaneously in a single dosage form, provided concomitantly in separate dosage forms, or provided in separate dosage forms for administration separated by some amount of time that is within the time in which both the compound of Formula I and the at least one additional active agent are within the blood stream of a patient. The compound of Formula I and the additional active agent need not be prescribed for a patient by the same medical care worker. The additional active agent or agents need not require a prescription. Administration of the compound of Formula I or the at least one additional active agent can occur via any appropriate route, for example, oral tablets, oral capsules, oral liquids, inhalation, injection, suppositories or topical contact.

The term “treatment”, as used herein, includes providing a compound of Formula I, either as the only active agent or together with at least one additional active agent sufficient to: (a) prevent a disease or a symptom of a disease from occurring in a patient who may be predisposed to the disease but has not yet been diagnosed as having it; (b) inhibiting the disease, i.e. arresting its development; and (c) relieving the disease, i.e., causing regression of the disease. “Treating” and “treatment” also means providing a therapeutically effective amount of a compound of Formula I, as the only active agent or together with at least one additional active agent to a patient suffering from schizophrenia, depression, stroke, or a neurodegenerative disease.

The term “therapeutically effective amount” of a pharmaceutical composition, as used herein, means an amount effective, when administered to a patient, to provide a therapeutic benefit such as an amelioration of symptoms, e.g., to treat a patient suffering from schizophrenia, depression, stroke, or a neurodegenerative disease.

The compounds may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, for example, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active agents may be included in a pharmaceutical composition, which do not substantially interfere with the activity of the compound of Formula I.

The pharmaceutical compositions can be formulated for oral administration. These compositions contain between 0.1 and 99 weight percent (“wt. %”) of a compound of Formula I and usually at least about 5 wt. %. Some embodiments contain from about 25 wt. % to about 50 wt. % or from about 5 wt. % to about 75 wt. % of a compound of Formula.

The pharmaceutical composition can be formulated in a package comprising the pharmaceutical composition of Formula I in a container and further comprising instructions for using the composition for the treatment of schizophrenia, depression, stroke, or a neurodegenerative disease.

In an embodiment, a method for treating schizophrenia, depression, stroke, or a neurodegenerative disease comprises providing an effective amount of a compound or salt of Formula I to a patient in need of such treatment. Alternatively, the compound may be provided in the form of a pharmaceutical composition.

The radiolabeled compounds of Formula I can be used to detect the presence and location of NR2B receptor subunits in an organ or body area, such as the brain or spinal cord, of a subject. The method comprises administration of a detectable quantity of a pharmaceutical composition containing a radiolabeled compound of Formula I or a pharmaceutically acceptable salt thereof, to a subject. A “detectable quantity” means that the amount of the compound that is administered is sufficient to enable detection of binding of the compound to the NR2B receptor subunit. An “imaging effective quantity” means that the amount of the compound that is administered is sufficient to enable imaging of the compound bound to the NR2B receptor subunit A “subject” is a human or non-human animal, specifically a human.

The radiolabeled compounds of Formula I are used in non-invasive nuclear medicine imaging techniques such as PET. Imaging is used to quantify NR2B receptor subunits in vivo. For nuclear medicine imaging, the radiation emitted from the organ or area being examined is measured and expressed either as total binding or as a ratio in which total binding in one tissue is normalized to (for example, divided by) the total binding in another tissue of the same subject during the same in vivo imaging procedure. Total binding in vivo is defined as the entire signal detected in a tissue by an in vivo imaging technique without the need for correction by a second injection of an identical quantity of labeled compound along with a large excess of unlabeled, but otherwise chemically identical compound.

For purposes of in vivo imaging, the compounds of Formula I are labeled. The type of detection is a major factor in selecting the label. For instance, labeling with ¹¹C and ¹⁸F are particularly suitable for in vivo PET imaging with the compounds of Formula I. The type of instrument used will guide the selection of the radionuclide or stable isotope. For instance, the radionuclide chosen should have a type of decay detectable by a given type of instrument. Another consideration relates to the half-life of the radionuclide. The half-life should be long enough so that it is still detectable at the time of maximum uptake by the target, but short enough so that the host does not sustain deleterious radiation. The radiolabeled compounds can be detected using nuclear medicine imaging wherein emitted radiation of the appropriate wavelength is detected.

The compounds of Formula I may be used to identify neurodegenerative processes in the brain. In an embodiment, imaging comprises PET imaging of the brain of a subject. Exemplary processes that can be studied include those associated with neurodegenerative or neuropsychiatric disorders such as stroke, epilepsy, dementia, traumatic brain injury, anxiety, schizophrenia, bipolar disorder, autism, HIV infection of the brain, Alzheimer's disease, mild cognitive impairment, Huntington's disease, Parkinson's disease, multiple sclerosis, psychosis, and depression, for example. In an embodiment, depression includes major depressive disorder. The labeled compounds of Formula I can be used for clinical investigation, diagnosis, and treatment.

Generally, the dosage of the labeled compounds will vary depending on considerations such as age, condition, sex, and extent of disease in the subject, contraindications, if any, concomitant therapies and other variables, to be adjusted by a physician skilled in the art. Dosage can vary from 0.001 μg/kg to 10 μg/kg, specifically 0.01 μg/kg to 1.0 μg/kg.

Administration to the subject can be local or systemic and accomplished intravenously, intra-arterially, intrathecally (via the spinal fluid) or the like. Administration can also be intradermal or intracavitary, depending upon the body site under examination. After administration of the radioligand, the area of the subject under investigation is examined by imaging techniques such as PET imaging techniques. The exact protocol can vary depending upon factors specific to the subject, as noted above, and depending upon the body site under examination, method of administration and type of label used; the determination of specific procedures would be routine to the skilled artisan. Blood sampling may accompany imaging to allow for measurement of the arterial input function of the radioligand. These PET and blood measurements can then be used by well-known biomathematical techniques to quantify NR2B density in areas of interest, in brain or spinal cord.

Examples of non-aqueous carriers are propylene glycol, polyethylene glycol, vegetable oil, and injectable organic esters such as ethyl oleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobials, anti-oxidants, chelating agents and inert gases. The pH and exact concentration of the various components of the pharmaceutical composition are adjusted according to routine skills in the art.

This invention is further illustrated by the following examples that should not be construed as limiting.

### EXAMPLES

Abbreviations used in the Examples below are set out in the following table.

| Term | Abbreviation |
|---|---|
| Acetonitrile | MeCN |
| Dimethyl formamide | DMF |
| High performance liquid chromatography | HPLC |
| 7-methoxy-2,3,4,5-tetrahydro-1H-benzo[d]azepin-1-ol | MTB |
| [¹¹C] 7-methoxy-3-(4-(4-(methylthio)phenyl)butyl)- | [¹¹C]NR2B-SMe |
| 2,3,4,5-tetrahydro-1H-benzo[d]azepin-1-ol) | |
| Positron emission tomography | PET |
| Room temperature | RT |
| Trifluoroacetic acid | TFA |

### Example 1. Synthesis of Derivatives Based on 7-methoxy-2,3,4,5-tetrahydro-1H-benzo[d]azepin-1-ol (MTB)

Derivatives based on MTB were synthesized in 1-4 steps in up to 40% overall yields.

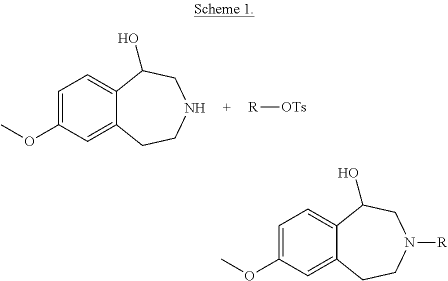

General procedure for synthesizing the derivatives (Scheme 1): 7-methoxy-2,3,4,5-tetrahydro-1H-benzo[d]azepin-1-ol, 1.0 g (5.2 mmol), R-OTs (1.2 eq), where R=L-Ar or a precursor thereof, Na₂HPO₄ (3 eq), were suspended in 5 mL anhydrous CH₃CN. The reaction mixture was heated at 75° C. overnight. The mixture was filtered to remove insoluble salts, and the product was purified using preparative HPLC portion-wise, using 0.025% TFA in water as solvent A and 0.025% TFA in CH₃CN as solvent 13. The gradient used was from 20% B to 95% B within 20 minutes using a C18 HPLC column at 30×250 mm (OD×L) with particle size of 10 μm at flow rate of 55 mL/min. The collected fractions were combined, and were added into a mixture of 100 mL H₂O and 100 mL CH₂C₂. After shaking vigorously, excess K)CO₃ solid was added, until the pH of the aqueous phase is at about pH=9. The organic phase was separated, and the aqueous phase was extracted 2 more times with 100 mL CH₂C₂ each. The combined CH₂C₂ phase was dried over MgSO₄. After filtration, the solvent was removed. The waxy material was dissolved in CH₃CN, and the solution was dried in Centrifan overnight. The chemical yield was 40-85%, depending the actual structure. If the collected HPLC fractions were dried using a rotary evaporator directly, the obtained compound was a TFA salt. ¹H and ¹³C{¹H} NMR spectra were recorded in CDCl₃, and the respective purity was determined using analytical HPLC.

All derivatives were evaluated for binding to the NR2B site and other neuro-targets. Table 1 provides the structures of the derivatives, molecular weight, calculated Log D values, the structure-activity relationship, and binding data.

TABLE 1

| | | | | | | σ₁ | σ₂ |
|---|---|---|---|---|---|---|---|
| | | | | | | receptor | receptor |
| | | | | Ki (nM), | Ki (nM), | at 10 | at 10 |
| # | Structure | Mw | cLogD | automatic | manual | μM (%) | μM (%) |
| 1 | 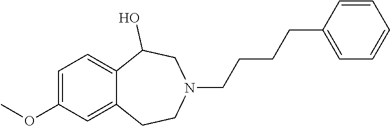 [SMILES: COc1ccc2c(c1)CCN(CCCCc1ccccc1)CC2O] | 439.48 | 3.1 | 43.5 | 9.8 | 89.1 | 87.8 |
| | | | | 5.4 ± 0.4* | 182 ± 38* | 554 ± 127* | |
| 2 | 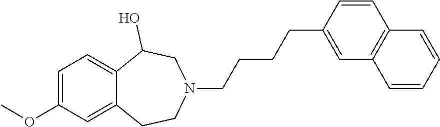 [SMILES: COc1ccc2c(c1)CCN(CCCCc1ccc3ccccc3c1)CC2O] | 489.54 | 4.3 | 22.7 | 7.3 | 84.4 | 94.0 |
| 3 | 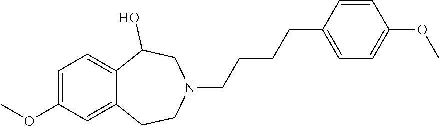 | 469.50 | 2.9 | 44.0 | 8.7 | 94.0 | 91.4 |
| 4 | 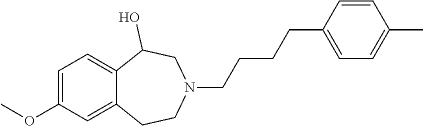 | 453.50 | 3.4 | 31.3 | 4.9 | 89.7 | 90.7 |
| 5 | 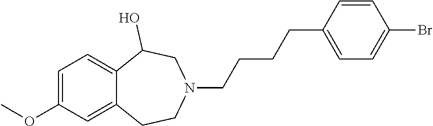 [SMILES: COc1ccc2c(c1)CCN(CCCCc1ccc(Br)cc1)CC2O] | 518.37 | 3.7 | 26.7 | 2.5 | 83.7 | 90.0 |
| 6 | 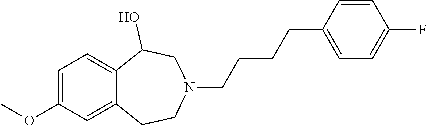 [SMILES: COc1ccc2c(c1)CCN(CCCCc1ccc(F)cc1)CC2O] | 457.47 | 3.1 | 28.0 | 3.7 | 57.9 | 76.9 |
| 7 | 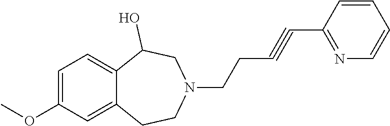 [SMILES: COc1ccc2c(c1)CCN(CCC#Cc1ccccn1)CC2O] | 436.43 | 1.9 | 493.0 | 97 | 66.4 | 74.2 |
| 8 | 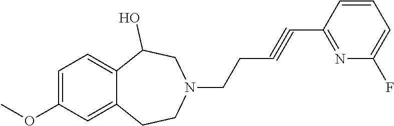 [SMILES: COc1ccc2c(c1)CCN(CCC#Cc1cccc(F)n1)CC2O] | 454.42 | 2.0 | 583.5 | 129 | 40.8 | 56.4 |
| 9 | 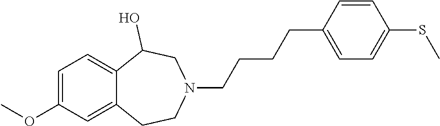 | 485.56 | 3.7 | 20.0 | | | |
| 10 | 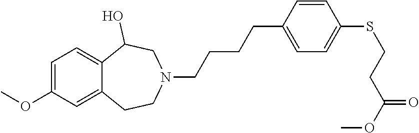 [SMILES: COC(=O)CCSc1ccc(CCCCN2CCc3cc(OC)ccc3C(O)C2)cc1] | 557.63 | 3.6 | 42.0 | | | |
| 11 | 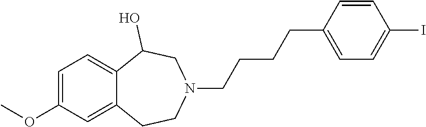 [SMILES: COc1ccc2c(c1)CCN(CCCCc1ccc(I)cc1)CC2O] | 565.37 | 4.0 | 21.0 | | | |
| 12 | 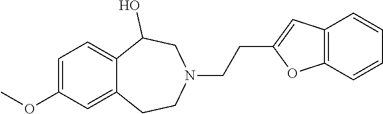 [SMILES: COc1ccc2c(c1)CCN(CCc1cc3ccccc3o1)CC2O] | 451.44 | 2.9 | 119.8 | | | |
| 13 | 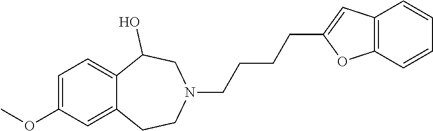 [SMILES: COc1ccc2c(c1)CCN(CCCCc1cc3ccccc3o1)CC2O] | 479.50 | 3.5 | 44.0 | | | |
| 14 | 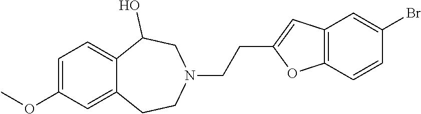 [SMILES: COc1ccc2c(c1)CCN(CCc1cc3cc(Br)ccc3o1)CC2O] | 530.34 | 3.7 | 66.3 | | | |
| 15 | 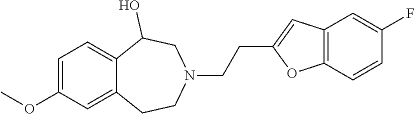 [SMILES: COc1ccc2c(c1)CCN(CCc1cc3cc(F)ccc3o1)CC2O] | 469.43 | 3.0 | 53.7 | | | |
| 16 | 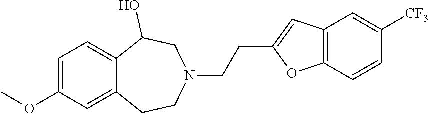 [SMILES: COc1ccc2c(c1)CCN(CCc1cc3cc(C(F)(F)F)ccc3o1)CC2O] | 519.44 | 3.7 | 48.7 | | | |
| 17 | 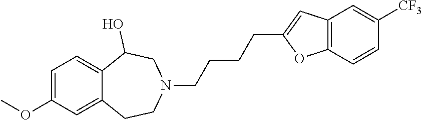 [SMILES: COc1ccc2c(c1)CCN(CCCCc1cc3cc(C(F)(F)F)ccc3o1)CC2O] | 547.49 | 3.9 | 19.3 | | | |

*Tewe B et al., ChemMedChem 2010, 5, 687.

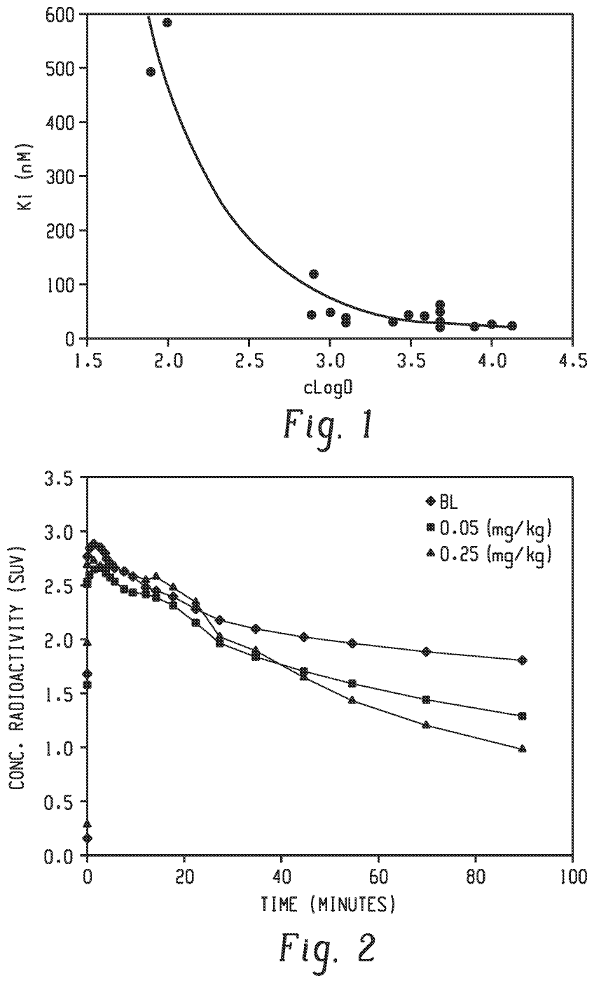

Results: Structure-activity relationship for derivatives based on MTB showed that the binding affinity of these compounds for the NR2B site is sensitive to substituents on the remote aryl group, either on the ring or attached. The groups most detrimental to binding are hydrophilic, such as pyridinyl. However, binding affinity does increase with molecular weight. Overall, the binding affinity versus calculated clogD tends to follow an exponential curve (FIG. 1), with other variables also affecting the binding. Binding affinity increases with lipophilicity. The tether length between the tertiary amine and the remote aryl group plays an important role, with a length of 4 methylene groups exhibiting good binding. Polar groups, such as ether, non-polar groups, such as thioether, or bulky groups, such as ketal were not optimal.

Conclusions: Structure-activity relationships were established for MTB derivatives. The PET imaging in rodents showed that these radioligands bind selectively and specifically to NR2B receptor, not preblocked by selective sigma1 receptor ligands. The most important factors for binding affinity based on the data set are hypophilicity and the length of the tether between the tertiary amine and the remote aryl group. The nature of the aryl ring also played a role.

### Example 2. Synthesis of [¹¹C] 7-methoxy-3-(4-(4-(4,4,5,5-tetramethyl-1,3,2-dioxaborolan-2-yl)phenyl)butyl)-2,3,4,5-tetrahydro-1H-benzo[d]azepin-1-ol ([¹¹C] NR2B-Me); PET Imaging in Rat

The precursor for labelling, 7-methoxy-3-(4-(4-(4,4,5,5-tetramethyl-1,3,2-dioxaborolan-2-yl)phenyl)butyl)-2,3,4,5-tetrahydro-1H-benzo[d]azepin-1-ol ([¹¹C]NR2B-Me), was synthesized in 4 steps. Treatment of this precursor (0.5 mg, 1.1 μmol) with cesium fluoride in methanol (1 M, 20 μL, 20 μmol), palladium catalyst (0.4 mg, 1:2 mixture of Pd₂(dba)₃, tris(2,4-dimethylphenyl)phosphine), and [¹¹C]Me in MeOH (400 μL) at 80° C. for 5 minutes gave [¹¹C]NR2B-Me, which was purified with HPLC on a Waters X-Bridge C18 column (250×10 mm) eluted with 0.1% TFA in H₂O/MeOH (50:50 v/v). [¹¹C]NR23-Me was obtained in 20% yield from cyclotron-produced [¹¹C]CO₂ and with a radiochemical purity of >99% and a mean molar activity of 180 GBq/μmol.

The purified [¹¹C]NR2B-Me was then formulated for intravenous injection. PET imaging of brain was performed after intravenous administration of [¹¹C]NR2B-Me to rats at baseline and after displacement with the NR2B-selective ligand Ro 25 6981 (4-[(1R,2S)-3-(4-Benzylpiperidin-1-yl)-1-hydroxy-2-methylpropyl]phenol) (at 0.01-3 mg/kg, i.v. at 10 min after radioligand injection).

Results: PET imaging of [¹¹C]NR2B-Me in rats at baseline revealed very high brain radioactivity uptake, reaching 3.0 SUV at 5 minutes followed by a slow washout over 90 minutes. Ro 25 6981 displacement accelerated brain radioactivity washout in a dose-dependent manner (FIG. 2).

Conclusions: [¹¹C]NR2B-Me was readily synthesized, and showed high brain uptake in rat, which could be displaced by Ro 25 6981.

### Example 3. Synthesis of [¹¹C] 7-methoxy-3-(4-(4-(methylthio)phenyl)butyl)-2,3,4,5-tetrahydro-1H-benzo[d]azepin-1-ol ([¹¹C]NR2B-SMe); PET Imaging in Rat

The precursor for labelling, methyl 3-((4-(4-(1-hydroxy-7-methoxy-1,2,4,5-tetrabydro-3H-benzo[d]azepin-3-yl)butyl)phenyl)thio)propanoate, was synthesized in 4 steps. The precursor was obtained in 38% overall yield. Treatment of this precursor (0.5 mg, 1.1 μmol) with tetrabutylammonium hydroxide (1 M, 5 μL, 5 μmol) and [¹¹C]MeI in DMF (400 μL) at RT for 5 minutes gave [¹¹C]NR2B-SMe (Scheme 2), which was purified with HPLC on a Waters X-Bridge C18 column (250×10 mm) eluted with 0.1% TFA in H₂O/MeCN (65:35 v/v). [¹¹C]NR2B-SMe was obtained in 20% yield from cyclotron-produced [¹¹C]CO₂ and with a radiochemical purity of >98% and a mean molar activity of 58 GBq/μmol.

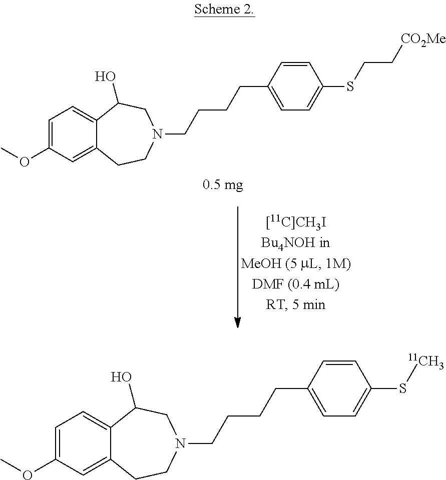

The purified [¹¹C]NR2B-SMe was then formulated for intravenous injection. PET imaging of brain was performed after intravenous administration of [¹¹C]NR2B-SMe to rats at baseline and preblocked with NR2B-SMe, the NR2B-selective ligands eliprodil or ifenprodil, or the sigma-1 receptor-selective compound, SA4503 (each at 0.01-3 mg/kg, i.v. at 15 min before radioligand injection). Displacement studies where NR2B-SMe or eliprodil was given at 15 min after radioligand were also performed.

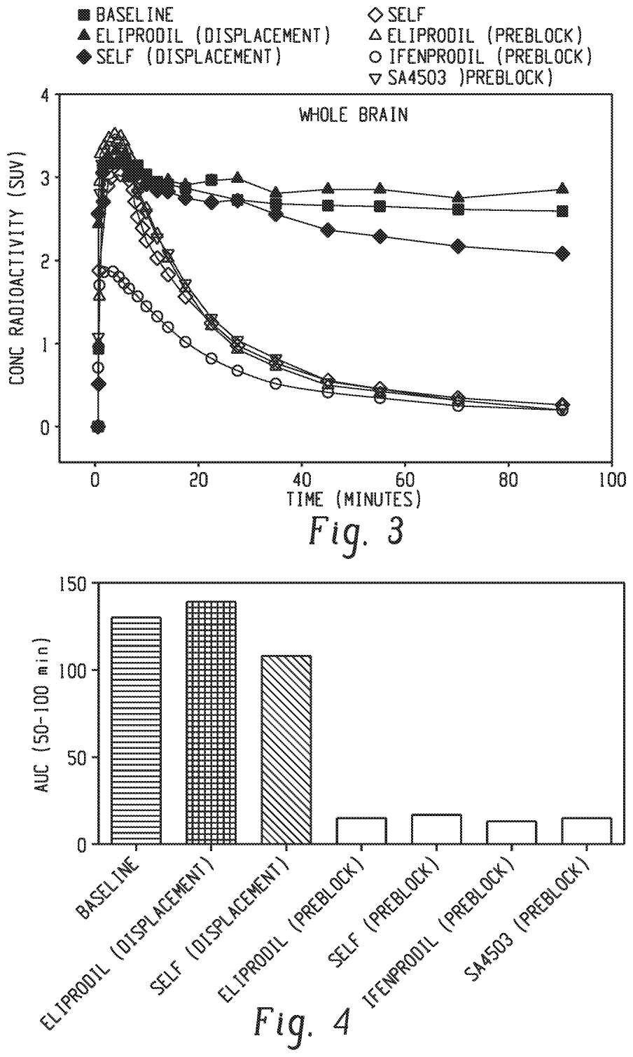

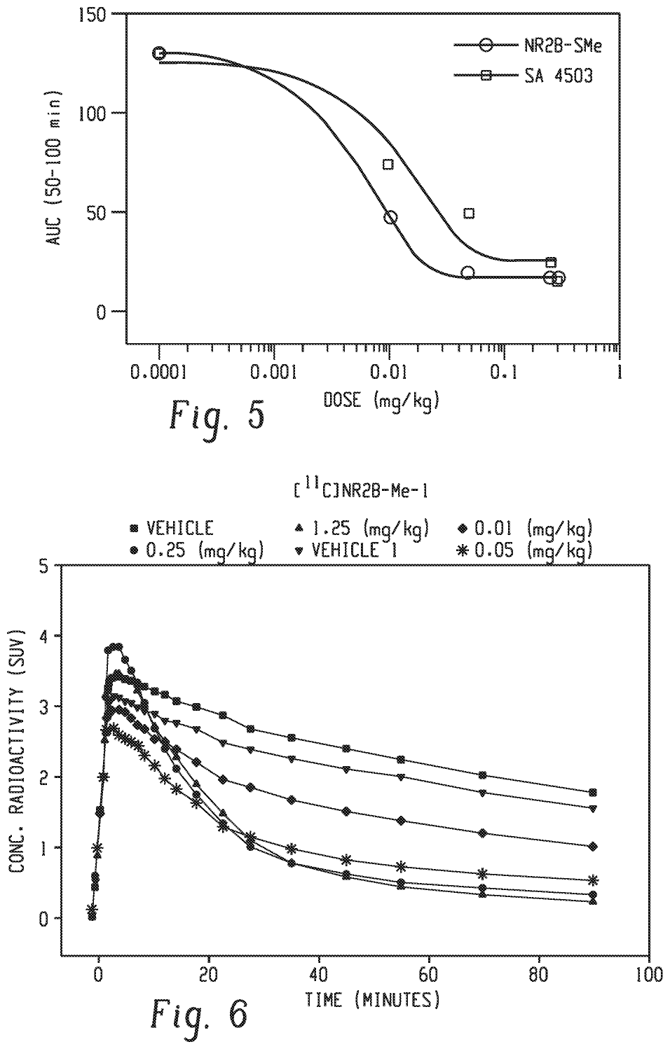

Results: PET imaging of [¹¹C]NR2B-SMe in rats at baseline revealed very high brain radioactivity uptake, reaching 3.5 SUV at 5 minutes followed by very low washout over 90 minutes. NR2B-SMe, and SA 4503 pretreatment blocked brain radioactivity uptake in a dose-dependent manner. FIG. 3 illustrates whole brain PET time activity curves in rats at baseline, rats pretreated with various agents and in rats given NR2B-SMe or eliprodil after radioligand. At high dose of pretreatment agent, a high proportion of brain radioactivity uptake (80%) was blocked. Eliprodil and ifendopril (3 mg/kg i.v) also blocked brain radioactivity uptake to the similar high extent. Eliprodil failed to displace radioligand, whereas NR2B-SMe gave only slow and low displacement over 90 min. FIG. 4 illustrates areas under the brain time-activity curve (AUC) between 50 and 100 min for the PET experiments in FIG. 3. FIG. 5 illustrates dependence of AUC on dose of NR2B-SMe and SA4503.

Conclusions: [¹¹C]NR2B-SMe was readily synthesized, and showed high brain uptake in rat, which could be pre-blocked by NR21-SMe itself, elipodil, ifenprodil, and by a selective sigma-1 receptor ligand. Because sigma-1 receptors serve as multitasking ion channel protein chaperones with involvement in modulation of NMDA activity, and are known to interact directly with the N-terminal domains of NR1 and NR2B subunits, further study will be conducted to characterize the nature of the specific binding of [¹¹C]NR2B-SMe in rat brain and its utility in brain research as a PET radioligand for NR2B subunits in NMDA receptors.

### Example 4. Synthesis of [¹¹C] (R)(−)-methoxy-3-(4-(4-(4,4,5,5-tetramethyl-1,3,2-dioxaborolan-2-yl)phenyl)butyl)-2,3,4,5-tetrahydro-1H-benzo[d]azepin-1-ol ([¹¹C] (R)(−)NR2B-Me; [¹¹C] NR2B-Me1); PET Imaging in Rat

The precursor for labelling, (R)(−)₇-methoxy-3-(4-(4-(4,4,5,5-tetramethyl-1,3,2-dioxaborolan-2-yl)phenyl)butyl)-2,3,4,5-tetrahydro-1H-benzo[d]azepin-1-ol, was prepared by separation of the racemic mixture using a chiral prep-HPLC column. Treatment of this precursor (0.5 mg, 1.1 μmol) with cesium fluoride in methanol (1 M, 20 μL, 20 μmol), palladium catalyst (0.4 mg, 1:2 mixture of Pd₂(dba)₃, tris(2,4-dimethylphenyl)phosphine), and [¹¹C]MeI in MeOH (400 μL) at 80° C. for 5 minutes gave [¹¹C]NR2B-MeI, which was purified with HPLC on a Waters X-Bridge C18 column (250×10 mm) eluted with 0.1% TFA in H₂O/MeOH (50:50 v/v). [¹¹C]NR2B-MeI was obtained in 20% yield from cyclotron-produced [¹¹C]CO₂ and with a radiochemical purity of >99% and a mean molar activity of 180 GBq/μmol

PET imaging of brain was performed after intravenous administration of [¹¹C]NR2B-MeI to rats at baseline and after displacement with the NR2B-selective ligand Ro 25 6981 (4-[(1R,2S)-3-(4-Benzylpiperidin-1-yl)-1-hydroxy-2-methylpropyl]phenol) (at 0.01-1.25 mg/kg, i.v. at 10 min after radioligand injection) (FIG. 6).

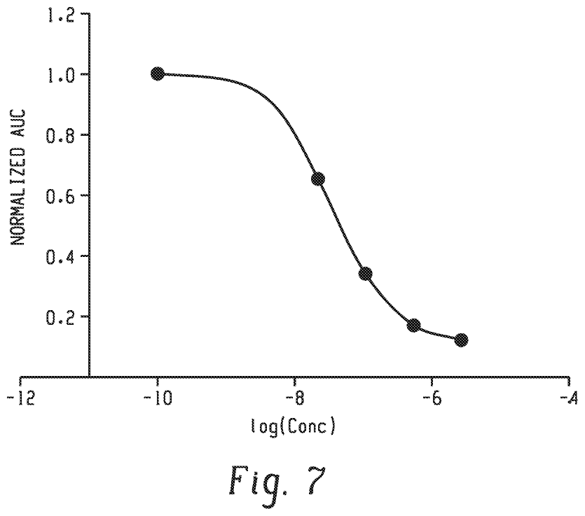

Results: PET imaging of [¹¹C]NR2B-MeI in rats revealed reversible binding with 95% preblocking and 65% displacement. The results showed very high brain radioactivity uptake within 5 minutes followed by a slow washout over 90 minutes. Ro 25 6981 displacement accelerated brain radioactivity washout in a dose-dependent manner (FIG. 6). FIG. 7 illustrates the dependence of AUC for [¹¹C]NR2B-MeI on the dose of Ro 25 6981.

Conclusions: [¹¹C]NR2B-MeI was readily synthesized, and showed high brain uptake in rat, which could be displaced by Ro 25 6981.

### Example 5. [¹¹C]NR2B-Me PET Imaging in Monkey

Brain PET Imaging (V_{T}/f_{P}) in monkey was performed after intravenous administration of [¹¹C]NR2B-MeI to monkeys at baseline, after displacement with Co101244 (1-[2-(4-hydroxyphenoxy)ethyl]-4-[(4-methylphenyl)methyl]-4-piperidinol), and after self-block. The binding potential of whole brain, BP_{ND} was 3.8 (Co101244) and 4.6 (self-block); V, showed time stability after 80 minutes of radioligand injection. Table 2 provides the normalized total volume of distribution (V_{T}/f_{P}) (ml/cm³) for whole brain and specific regions of the brain at baseline, Co101244 (1-[2-(4-hydroxyphenoxy)ethyl]-4-[(4-methylphenyl)methyl]-4-piperidinol) block and self-block.

TABLE 2
Normalized Total Volume of Distribution (V_{T}/fₚ) (ml/cm³)

| | Baseline | | | |
|---|---|---|---|---|
| | Ave. | SD | Co101244 | Self-block |
| Whole Brain | 1959.7 | 406.6 | 511.6 | 408.1 |
| Frontal Cortex | 2254.5 | 458.5 | 562.0 | 451.4 |
| Cingulate Cortex | 2388.9 | 407.3 | 523.3 | 400.0 |
| Striatum | 1965.2 | 402.9 | 573.6 | 437.8 |
| Insula | 2314.2 | 508.9 | 542.6 | 408.1 |
| Temporal Lobe | 1956.8 | 390.8 | 476.7 | 373.0 |
| Amygdala | 3171.1 | 1256.7 | 515.5 | 405.4 |
| Hippocampus | 2389.6 | 692.7 | 465.1 | 370.3 |
| Thalamus | 2385.1 | 675.3 | 596.9 | 445.9 |
| Parietal Lobe | 2001.4 | 398.6 | 538.8 | 440.5 |
| Occipital Lobe | 1676.3 | 398.9 | 484.5 | 402.7 |
| Cerebellum | 1465.9 | 303.6 | 426.4 | 348.6 |

The compounds, compositions, and methods disclosed herein include(s) at least the following aspects:

Aspect 1: A compound of Formula I, or a radioligand thereof, and/or a pharmaceutically acceptable salt thereof:

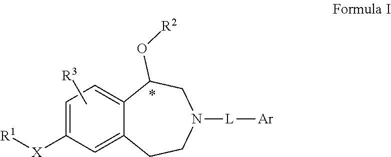

wherein X is O or S, specifically O; R is H C₁-C₆ alkyl, C₁-C₆ haloalkyl, specifically C₁-C₃ alkyl, and more specifically methyl; R² is H, C₁-C₆ alkyl, C₁-C₆ haloalkyl, specifically 1 or C₁-C₃alkyl, and more specifically H; R is H, C₁-C₆ alkyl, C₁-C₆ alkoxy, C₁-C₆ haloalkyl, C₁-C₆ haloalkoxy, —OH, halo, —COOH, —CN, —NO₂, amino, mono- or di-alkylamino, C₂-C₆ alkanoyl, C₂-C₈ cycloalkyl, or heterocycloalkyl, specifically H, C₁-C₆ alkyl, or C₂-C₆ haloalkyl, and more specifically H; L is a linking group; and Ar is an aryl or heteroaryl group, each of which is optionally substituted with one, two, or three substituents; with the provision that the compound is not 3-(4-phenylbutyl)-2,3,4,5-tetrahydro-1H-benzo[d]azepine-1,7-diol; 7-methoxy-3-(4-phenylbutyl)-2,3,4,5-tetrahydro-1H-benzo[d]azepin-1-ol; or 7-[11C]methoxy-3-(4-phenylbutyl)-2,3,4,5-tetrahydro-1H-benzo[d]azepin-1-ol.

Aspect 2: The compound of Aspect 1, wherein the compound is a radioligand having an atom of Formula I selected from carbon, hydrogen, nitrogen, oxygen and halogen atom that comprises, or is replaced by, a detectable amount of ¹¹C, ¹³N, ¹⁵O, ¹⁸F, ⁷⁵Br, or ⁷⁶Br.

Aspect 3: The compound of any one of Aspect 1-2, wherein the linking group L is an optionally substituted divalent C₂-C₈ alkyl, C₂-C₈ alkenyl, or C₂-C₈ alkynyl, specifically a divalent C₂-C₆ alkyl, C₂-C₆ alkenyl, or C₂-C₆ alkynyl, more specifically a divalent C₂-C₄ alkyl, C₂-C₄ alkenyl, or C₂-C₄ alkynyl, and yet more specifically a divalent C₃-C₄ alkyl; wherein when optionally substituted, each substituent independently is oxo, C₁-C₆ alkyl, C₁-C₆ alkoxy, C₁-C₆ haloalkyl, C₁-C₆ haloalkoxy, —OH, halo, —COOH, —CN, —NO₂, amino, mono- or di-alkylamino, C₂-C₆alkanoyl, C₂-C₈ cycloalkyl, or heterocycloalkyl; or wherein the linking group chain is substituted with one, two, or three heteroatoms between carbon atoms within the linking group chain, the heteroatoms selected from O, S, S═O, S(═O)₂, or NR wherein R is H, C₁-C₆ alkyl, C₁-C₆ haloalkyl, or C₂-C₆ alkanoyl.

Aspect 4: The compound of any one of Aspects 1-3, specifically Aspects 1-2, wherein the Ar group of Formula I is phenyl, naphthyl, bi-phenyl, pyridyl, benzofuranyl, coumarinyl, quinolinyl, isoquinolinyl, quinazolinyl, pyridizinyl, pyrazinyl, pyrimidinyl, furanyl, oxazolyl, pyrrolyl, thienyl, thiazolyl, triazinyl, triazolyl, tetrazolyl, isoxazolyl, imidazolyl, indolyl, benz[b]thiophenyl benzothiazolyl, pyrazolyl, isoquinolinyl, quinazolinyl, quinoxalinyl, or isoindolyl, each of which is optionally substituted with one, two, or three substituents individually selected from C₁-C₆ alkyl, C₁-C₆ alkoxy, C₁-C₆ thioalkyl, C₁-C₆ haloalkyl, C₁-C₆ haloalkoxy, —OH, halo, —COOH, —CN, —NO, amino, mono- or di-alkylamino, C₂-C₆ alkanoyl, C₂-C₈ cycloalkyl, heterocycloalkyl, (C₁-C₆alkoxycarbonyl)C₀-C₆alkyl, (C₁-C₆alkoxycarbonyl)C₀-C₆alkoxy, or (C₁-C₆alkoxycarbonyl)C₀-C₆thioalkyl groups, specifically C₁-C₆ alkyl, C₁-C₆ alkoxy, C₁-C₆ thioalkyl, C₁-C₆ haloalkyl, halo, or (C₁-C₆alkoxycarbonyl)C₀-C₆thioalkyl.

Aspect 5: The compound of any one of Aspects 1-4, specifically Aspects 1-2, wherein X is O; R is C₁-C₃ alkyl; R² is H or C₁-C₃ alkyl; R³ is H, C₁-C₃ alkyl, or C₁-C₃ haloalkyl; L is a divalent C₂-C₆alkyl, C₂-C₆ alkenyl, or C₂-C₆ alkynyl; and Ar is phenyl, naphthyl, pyridyl, or benzofuranyl, each of which is optionally substituted with one substituent, wherein the substituent is C₁-C₆ alkyl, C₁-C₆ alkoxy, C₁-C₆ thioalkyl, —C₆ haloalkyl, halo, or (C₁-C₆ alkoxycarbonyl)C₀-C₆ thioalkyl.

Aspect 6: The compound of any one of Aspects 1-2, wherein the compound is

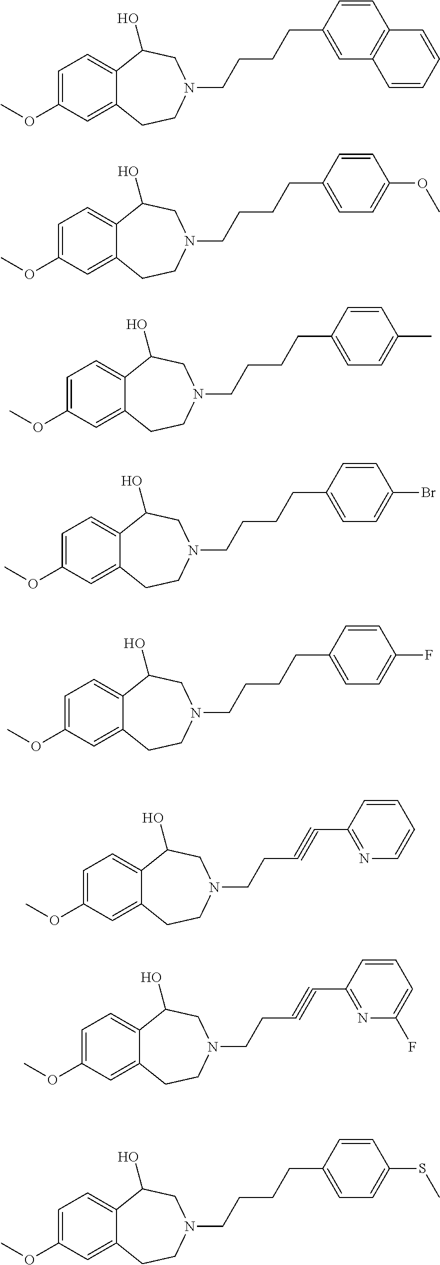

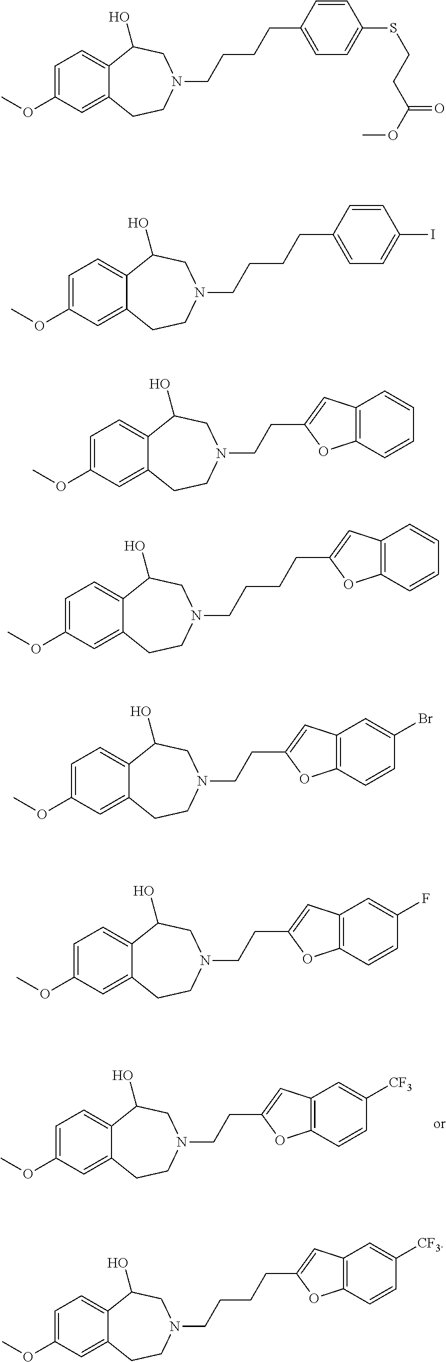

Aspect 7: The compound of any one of Aspects 1-2, wherein the compound is 7-methoxy-3-(4-(4-methylphenyl)butyl)-2,3,4,5-tetrahydro-1H-benzo[d]azepin-1-ol (NR2B-Me); (R)(−)7-methoxy-3-(4-(4-methylphenyl)butyl)-2,3,4,5-tetrahydro-1H-benzo[d]azepin-1-ol; 7-methoxy-3-(4-(4-(methylthio)phenyl)butyl)-2,3,4,5-tetrahydro-1H-benzo[d]azepin-1-ol (NR2B-SMe); (R)-7-methoxy-3-(4-(4-(methylthio)phenyl)butyl)-2,3,4,5-tetrahydro-1H-benzo[d]azepin-1-ol (NR2B3-SMe); or a [¹¹C]-labeled derivative thereof.

Aspect 8: The compound of any one of Aspects 1-6, specifically Aspects 1-2, wherein the stereogenic center * is in the R configuration.

Aspect 9: The compound of any one of Aspects 1-6, specifically Aspects 1-2, wherein the stereogenic center * is in the S configuration.

Aspect 10: A pharmaceutical composition comprising a compound or salt of any one of Aspects 1-9, specifically Aspects 1-2, and at least one pharmaceutically acceptable carrier.

Aspect 11: The pharmaceutical composition of Aspect 10, wherein the composition is formulated as an injectable fluid, an aerosol, a cream, a gel, a tablet, a pill, a capsule, a syrup, an ophthalmic solution, or a transdermal patch.

Aspect 12: A package comprising the pharmaceutical composition of Aspect 10 or 11 in a container and further comprising instructions for using the composition in order to treat a patient suffering from schizophrenia, depression, stroke, or a neurodegenerative disease, especially neuropain.

Aspect 13: A method for treating schizophrenia, depression, stroke, or a neurodegenerative disease, especially neuropain, comprising providing a therapeutically effective amount of a compound or salt of any one of Aspects 1-9, specifically Aspects 1-2, to a patient in need of such treatment.

Aspect 14: A method for treating schizophrenia, depression, stroke, or a neurodegenerative disease, especially neuropain, comprising providing a therapeutically effective amount of a pharmaceutical composition of Aspect 10 or 11 to a patient in need of such treatment.

Aspect 15: A method for quantifying NR2B receptor subunits within NMDA receptors in a subject comprises, administering a radiolabeled compound of any one of Aspects 1-9, specifically Aspects 1-2, to a subject, and quantifying the concentration of the radiolabeled compound using positron emission tomography.

The terms “a” and “an” do not denote a limitation of quantity, but rather denote the presence of at least one of the referenced item. The term “or” means “and/or”. Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments. The modifier “about” used in connection with a quantity is inclusive of the stated value and has the meaning dictated by the context (e.g., includes the degree of error associated with measurement of the particular quantity).

The endpoints of all ranges directed to the same component or property are inclusive of the endpoints, are independently combinable, and include all intermediate points and ranges (e.g., ranges of “up to about 25 wt. %, or, more specifically, about 5 wt. % to about 20 wt. %,” is inclusive of the endpoints and all intermediate values of the ranges of “about 5 wt. % to about 25 wt. %,” such as about 10 wt % to about 23 wt %, etc.).

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A compound of Formula I, or a radioligand thereof, and/or a pharmaceutically acceptable salt thereof: 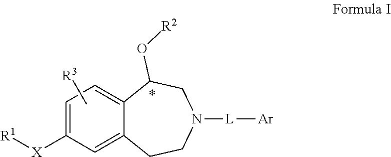
wherein
X is O or S, specifically 0;
R¹ is H, C₁-C₆ alkyl, C₁-C₆ haloalkyl, specifically C₁-C₃ alkyl, and more specifically methyl;
R² is H, C₁-C₆ alkyl, C₁-C₆ haloalkyl, specifically H or C₁-C₃ alkyl, and more specifically H;
R³ is H, C₁-C₆ alkyl, C₁-C₆ alkoxy, C₁-C₆ haloalkyl, C₁-C₆ haloalkoxy, —OH, halo, —COOH, —CN, —NO₂, amino, mono- or di-alkylamino, C₂-C₆ alkanoyl, C₂-C₈ cycloalkyl, or heterocycloalkyl, specifically H, C₁-C₃ alkyl, or C₁-C₃ haloalkyl, and more specifically H;
L is a linking group; and
Ar is an aryl or heteroaryl group, each of which is optionally substituted with one, two, or three substituents;
with the provision that the compound is not 3-(4-phenylbutyl)-2,3,4,5-tetrahydro-1H-benzo[d]azepine-1,7-diol; 7-methoxy-3-(4-phenylbutyl)-2,3,4,5-tetrahydro-1H-benzo[d]azepin-1-ol; or 7-[11C]methoxy-3-(4-phenylbutyl)-2,3,4,5-tetrahydro-1H-benzo[d]azepin-1-ol.

2. The compound of claim 1, wherein the compound is a radioligand having an atom of Formula I selected from carbon, hydrogen, nitrogen, oxygen and halogen atom that comprises, or is replaced by, a detectable amount of ¹¹C, ¹³N, ¹⁵O, ¹⁸F, ⁷⁵Br, or ⁷⁶Br.

3. The compound of claim 1, wherein the linking group L is an optionally substituted divalent C₂-C₈ alkyl, C₂-C₈ alkenyl, or C₂-C₈ alkynyl, specifically a divalent C₂-C₆ alkyl, C₂-C₆ alkenyl, or C₂-C₆ alkynyl, more specifically a divalent C₂-C₄ alkyl, C₂-C₄ alkenyl, or C₂-C₄ alkynyl, and yet more specifically a divalent C₃-C₄ alkyl;
wherein when optionally substituted, each substituent independently is oxo, C₁-C₆ alkyl, C₁-C₆ alkoxy, C₁-C₆ haloalkyl, C₁-C₆ haloalkoxy, —OH, halo, —COOH, —CN, —NO₂, amino, mono- or di-alkylamino, C₂-C₆ alkanoyl, C₂-C₈ cycloalkyl, or heterocycloalkyl;
or wherein the linking group chain is substituted with one, two, or three heteroatoms between carbon atoms within the linking group chain, the heteroatoms selected from O, S, S═O, S(═O)₂, or NR wherein R is H, C₁-C₆ alkyl, C₁-C₆ haloalkyl, or C₂-C₆ alkanoyl.

4. The compound of claim 1, wherein the Ar group of Formula I is phenyl, naphthyl, bi-phenyl, pyridyl, benzofuranyl, coumarinyl, quinolinyl, isoquinolinyl, quinazolinyl, pyridizinyl, pyrazinyl, pyrimidinyl, furanyl, oxazolyl, pyrrolyl, thienyl, thiazolyl, triazinyl, triazolyl, tetrazolyl, isoxazolyl, imidazolyl, indolyl, benz[b]thiophenyl benzothiazolyl, pyrazolyl, isoquinolinyl, quinazolinyl, quinoxalinyl, or isoindolyl, each of which is optionally substituted with one, two, or three substituents individually selected from C₁-C₆ alkyl, C₁-C₆ alkoxy, C₁-C₆ thioalkyl, C₁-C₆ haloalkyl, C₁-C₆ haloalkoxy, —OH, halo, —COOH, —CN, —NO₂, amino, mono- or di-alkylamino, C₂-C₆ alkanoyl, C₂-C₈ cycloalkyl, heterocycloalkyl, (C₁-C₆alkoxycarbonyl)C₀-C₆alkyl, (C₁-C₆alkoxycarbonyl)C₀-C₆alkoxy, or (C₁-C₆alkoxycarbonyl)C₀-C₆thioalkyl groups, specifically C₁-C₆ alkyl, C₁-C₆ alkoxy, C₁-C₆ thioalkyl, C₁-C₆ haloalkyl, halo, or (C₁-C₆alkoxycarbonyl)C₀-C₆thioalkyl.

5. The compound of claim 1, wherein
X is O;
R¹ is C₁-C₃ alkyl;
R² is H or C₁-C₃ alkyl;
R³ is H, C₁-C₃ alkyl, or C₁-C₃ haloalkyl;
L is a divalent C₂-C₆ alkyl, C₂-C₆ alkenyl, or C₂-C₆ alkynyl; and
Ar is phenyl, naphthyl, pyridyl, or benzofuranyl, each of which is optionally substituted with one substituent, wherein the substituent is C₁-C₆ alkyl, C₁-C₆ alkoxy, C₁-C₆ thioalkyl, C₁-C₆ haloalkyl, halo, or (C₁-C₆ alkoxycarbonyl)C₀-C₆ thioalkyl.

6. The compound of claim 1, wherein the compound is

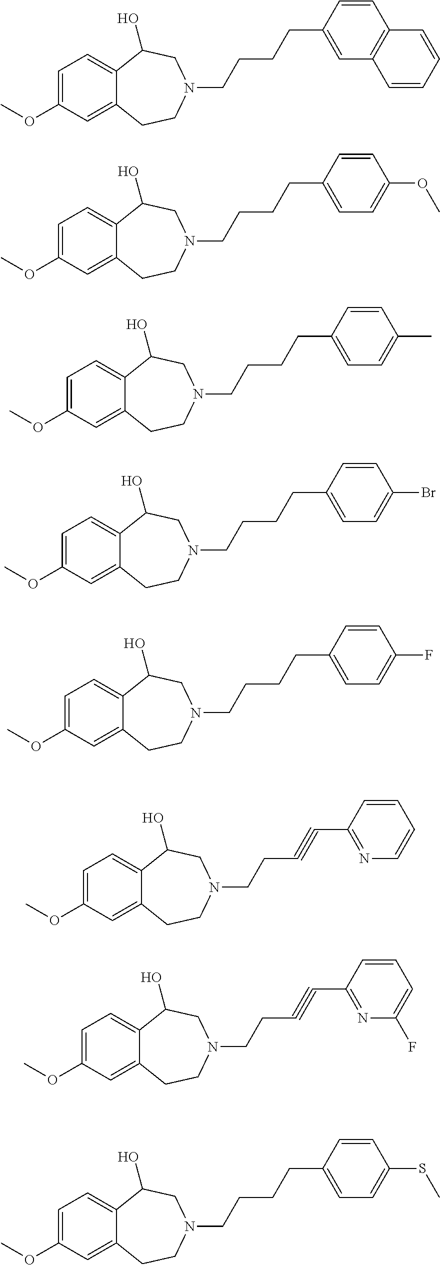

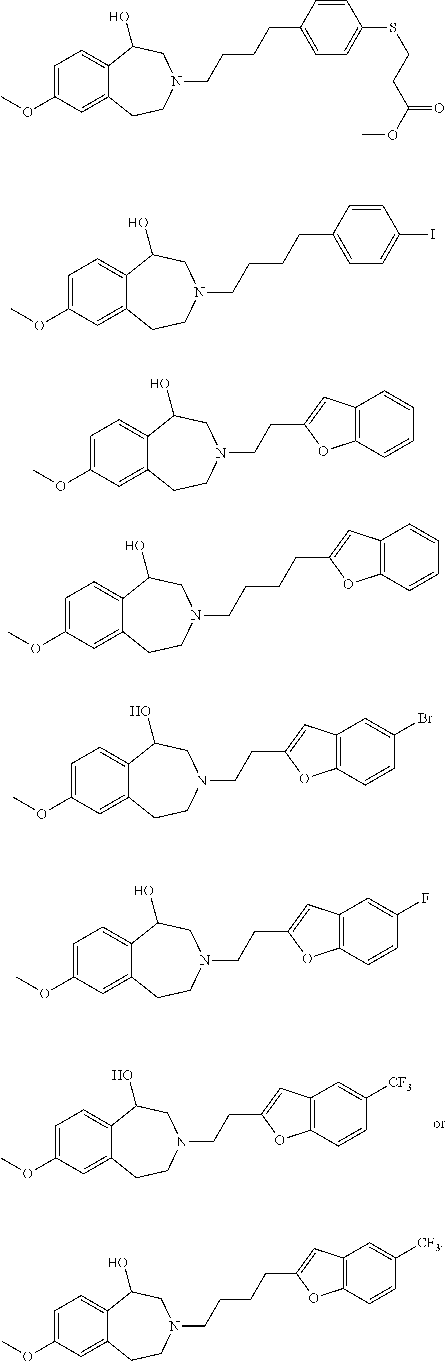

7. The compound of claim 1, wherein the compound is 7-methoxy-3-(4-(4-methylphenyl)butyl)-2,3,4,5-tetrahydro-1H-benzo[d]azepin-1-ol (NR2B-Me); (R)(−)₇-methoxy-3-(4-(4-methylphenyl)butyl)-2,3,4,5-tetrahydro-1H-benzo[d]azepin-1-ol; 7-methoxy-3-(4-(4-(methylthio)phenyl)butyl)-2,3,4,5-tetrahydro-1H-benzo[d]azepin-1-ol (NR2B-SMe); (R)-7-methoxy-3-(4-(4-(methylthio)phenyl)butyl)-2,3,4,5-tetrahydro-1H-benzo[d]azepin-1-ol (NR2B-SMe); or a [¹¹C]-labeled derivative thereof.

8. The compound of claim 1, wherein the stereogenic center * is in the R configuration.

9. The compound of claim 1, wherein the stereogenic center * is in the S configuration.

10. A pharmaceutical composition comprising a compound or salt of claim 1 and at least one pharmaceutically acceptable carrier.

11. The pharmaceutical composition of claim 10, wherein the composition is formulated as an injectable fluid, an aerosol, a cream, a gel, a tablet, a pill, a capsule, a syrup, an ophthalmic solution, or a transdermal patch.

12. A package comprising the pharmaceutical composition of claim 10 in a container and further comprising instructions for using the composition in order to treat a patient suffering from schizophrenia, depression, stroke, or a neurodegenerative disease, especially neuropain.

13. A method for treating schizophrenia, depression, stroke, or a neurodegenerative disease, especially neuropain, comprising providing a therapeutically effective amount of a compound or salt of claim 1 to a patient in need of such treatment.

14. A method for treating schizophrenia, depression, stroke, or a neurodegenerative disease, especially neuropain, comprising providing a therapeutically effective amount of a pharmaceutical composition of claim 10 to a patient in need of such treatment.

15. A method for quantifying NR2B receptor subunits within NMDA receptors in a subject comprises, administering a radiolabeled compound of claim 1 to a subject, and quantifying the concentration of the radiolabeled compound using positron emission tomography.
